(19) United States
(12) Patent Application Publication
(10) Pub. No.: US 2025/0375416 A1
(43) Pub. Date: Dec. 11, 2025

(54) SERINE PROTEASE INHIBITORY EFFECTS OF HEALTH SUPPLEMENTS IN HUMAN CANCEROUS CELL LINES

(71) Applicant: Teerapol SRICHANA, Hat Yai, Songkhla (TH)

(72) Inventors: Teerapol SRICHANA, Hat Yai, Songkhla (TH); Kumar Bijon SIL, Hat Yai, Songkhla (TH); Kumar Pijush PAUL, Hat Yai, Songkhla (TH)

(21) Appl. No.: 18/876,617

(22) PCT Filed: Dec. 25, 2023
(86) PCT No.: PCT/TH2023/000029

§ 371 Date: Dec. 18, 2024

(30) Foreign Application Priority Data
Dec. 27, 2022 (SG) 10202260606X

(51) Int. Cl. A61K 31/375; A61K 9/00; A61K 9/20; A61K 31/4415; A61K 31/51

(52) U.S. Cl. CPC A61K 31/375; A61K 9/0053; A61K 9/20; A61K 31/4415; A61K 31/51

(57) ABSTRACT

The present invention relates generally to a method of detecting inhibitory effects of health supplements on serine protease in cell lines derived from human carcinomas. More, particularly, the present invention relates to inhibitory analysis of health supplementary products taken from market and/or chemically purified forms, in order to determine their competitors and/or inhibitory effects on serine protease such as trypsin so that these supplements can be used for the treatment once someone develop intense inflammatory reactions either due to infections like COVID-19 and/or pathological conditions such as cancer. The present invention further provides therapeutic applications for agents and/or condition which utilize human enzymatic system and/or cellular components for the development of pathological conditions.

### FIELD OF THE INVENTION

This disclosure relates to a method of detecting inhibitory effects of health supplements on serine protease in cell lines derived from human carcinomas.

### BACKGROUND OF THE INTERVENTION

Introduction of Inflammation is a physiological part of the complex biological response of tissues to counteract various harmful signals. This process involves diverse actors such as immune cells, cytokines, blood vessels, and nerves as sources of orchestrating the development and control of inflammatory activities. Among them serine proteases are key elements in both physiological and pathological inflammation. Areas covered Serine protease inhibitors to treat inflammatory diseases are being actively investigated by various industrial and academic institutions. Expert opinion says Serine proteases regulating inflammation are versatile enzymes, usually involved in proinflammatory cytokine production and activation of immune cells. Their dysregulation during inflammation can have devastating consequences, promoting various diseases including skin and lung inflammation, neuroinflammation, and inflammatory arthritis. Several serine proteases were selected for contribution to inflammatory diseases and significant efforts that are spread to develop inhibitors. Strategies developed for inhibitor identification consist on either peptide-based inhibitor derived from endogenous protein inhibitors or small-organic molecules. It is also worth noting that among the recent patents on serine protease inhibitors related to inflammation a significant number are related to retinal vascular dysfunction and skin diseases. The present patent of serine protease inhibitors is non-protein and/or peptide-based FDA approved health supplements for the potential therapeutic application of inflammatory diseases.

Serine proteases (SPs) are distributed among all living cells. It accounts for almost one-third of all proteases, and serve as inevitable components in catalysing hydrolytic reactions both intra- and extracellularly. SPs participate in various physiological processes such as food digestion, embryo development and immune Défense (1). Although SPs are of great importance, sometimes they may be potentially hazardous to their enzymatic environments when not being properly controlled (2). The hazard of excessive peptidase activities includes coagulation, melanisation, tissue damage, etc (3,4). In fact, the action of SP s is tightly regulated by their inhibitors, including serine protease inhibitors (5, 6,7). The presences of nucleophilic Serine residue in the active site that attacks the carbonyl moiety of substrate, derives its name as serine protease (FIGS. 1 & 2). The Sps are endoproteases and catalyse polypeptide bond hydrolysis in the middle of the chain and serve to degrade invading organisms, antigen-antibody complexes and certain tissue proteins which are no longer necessary or useful for the body.

One of the proteases that is of particular pharmacological interest is leukocyte elastase. Leukocyte elastase, when released extracellularly, degrades connective tissue and other valuable proteins. While it is necessary for a normally functioning organism to degrade a certain amount of connective tissue and other proteins, the presence of an excessive amount of leukocyte elastase has been associated with various pathological states, such as emphysema and rheumatoid arthritis. To counteract the effects of leukocyte elastase when it is present in amounts higher than normal, a protease inhibitor has been sought which is specific for leukocyte elastase. Such a protease inhibitor would be especially useful if it were capable of being prepared from health regulatory approved chemicals in a purified form and in sufficient quantities to be pharmaceutically useful.

In a normally functioning organism, proteolytic enzymes are produced in a limited quantity and are regulated in part through the synthesis of protease inhibitors. When SPs finish their work, they are inactivated by SPIs and then move out of the circulation, to keep the homeostasis of living body. The roles of SPIs include blood coagulation, reproduction, complement system and innate immune response are well documented (8,9). Protease signalling pathways are stringently controlled, and deregulation of proteolytic activity results in the degradation of extracellular matrix which plays a major role in cancer progression.

A large number of naturally occurring protease inhibitors serve to control the endogenous proteases by limiting their reactions locally and temporally. In addition, the protease inhibitors may inhibit proteases introduced into the body by infective agents. Tissues that are particularly prone to proteolytic attack and infection, e.g., those of the respiratory and gastrointestinal tracts, are rich in protease inhibitors.

Protease inhibitors comprise approximately 10% of the human plasma proteins. At least eight inhibitors have been isolated from this source and characterized in the literature. These include α2-macroglobulin (α2M), α1-protease inhibitor (α1 PI), α1-antichymotrypsin (α1Achy), β1-anticollagenase (β1Ac), and inter-a-trypsin inhibitor (|α|).

A disturbance of the protease/protease inhibitor balance can lead to protease-mediated tissue destruction, including emphysema, arthritis, glomerulonephritis, periodontitis, muscular dystrophy, tumor invasion and various other pathological conditions. In certain situations, e.g., severe pathological processes such as sepsis or acute leukemia, the amount of free proteolytic enzymes present increases due to the release of enzyme from the secretory immune cells.

In patients where such aberrant conditions are present, serious damage to the body physiology can occur unless measures can be taken to control the proteolytic enzymes. Therefore, protease inhibitors have been needed which are capable of being administered to the body to control the proteolytic enzymes.

One protease that is of particular pharmacological interest is leukocyte elastase. Leukocyte elastase, when released extracellularly, degrades connective tissue and other valuable proteins. While it is necessary for a normally functioning physiology to degrade a certain amount of connective tissue and other proteins, the presence of an excessive amount of leukocyte elastase has been associated with various pathological states, such as emphysema and rheumatoid arthritis. To counteract the effects of leukocyte elastase when it is present in amounts greater than normal, protease inhibitors are available and specific for leukocyte elastase. Serine proteases, serine protease inhibitors, and protease-activated receptors have been intensively investigated and their roles in a wide range of processes—complement cascade, coagulation, inflammation, and digestion, for example—have been well characterized. However, if protease inhibitors would be especially useful from approved health supplement without side-effects and commonly available could be a pharmaceutically useful and well accepted at all level of health care system.

Trypsin is another potent protease of particular interest from a pharmacological standpoint. Trypsin is known to initiate degradation of certain soft organ tissue, such as pancreatic tissue, during a variety of acute conditions, such as pancreatitis. A variety of efforts have been directed toward the treatment of these conditions, without marked success, through the use of proteins which it was hoped would inhibit the action of trypsin. Illustrative of such efforts are attempts to use exogenous bovine trypsin inhibitors in treatment of human pancreatitis. While such techniques have been attempted in Europe, they have not been approved as effective by the U.S. Food and Drug Administration. Thus, there is a need for a protease inhibitor effective in neutralizing excess trypsin in a variety of acute and chronic conditions. As was the case with the leukocyte elastase inhibitor discussed above, a trypsin inhibitor would be particularly useful if it could be available from health supplements which are already in market and approved by FDA for general population.

Collagenase is an enzyme that breaks down collagen in damaged tissues within the skin and helps the body generate new healthy tissue. It is considered a virulence factor, facilitating the spread of gas gangrene. Collagen is a type of protein that connects and supports fibers in body tissues such as skin, tendons, muscles, and bone.

Leukocyte elastase, trypsin, cathepsin G and pancreatic elastase are examples of a class of proteases known as serine proteases, which have elements of common structure and mechanism. Their activity against different substrates and their sensitivity to different inhibitors are believed to result from changes in only a few amino acid residues. By analogy, it is possible to conceive of a class of serine protease inhibitors, also having common elements of structure and mechanism, in which changes in a relatively few amino acids will result in inhibition of different proteases, and that at least one member of this class will inhibit every serine protease of the former class. The class of serine protease inhibitors would then be of substantial value.

The serine protease inhibitors comprise a large family of molecules involved in inflammatory responses, blood clotting, and complement activation. The gene expression of two of the serine protease inhibitors, SPI 2.1 and 2.2, is tightly controlled by growth hormone in rat liver. Serpins are glycoproteins consisting of a central chain of 350-500 amino acids and several carbohydrate side chains, folding into 3 β-sheets and 8-9 α-helices, which hold a mobile loop of 20 residues that support the active site of the enzyme which serves as a substrate to the catalytic triad of serine proteases. Currently, numbers of serine protease inhibitors have been identified in the literature (10). However, non-protein-based serine protease inhibitors are not yet being reported which could be better in terms side-effects that be could induced due to immunological reactions following multiple applications.

Protease inhibitors tend to compete with substrate binding, either through direct competition or deformation of the protease active site. While protein inhibitors can gain potency through the burial of a large surface area and specificity through contacts with specific ecosites, small molecule inhibitors primarily gain potency through interactions with the catalytic machinery of the enzyme, and specificity through interactions with the substrate binding sites. While there are several examples of successful small molecule protease inhibitors in the clinic, selectivity and potency can be significant challenges when targeting particular protease family members. The search for novel modes of enzyme control, such as allosteric regulation, is therefore particularly exciting, with the hope that these regulatory sites will be more amenable to the design of specific and efficacious inhibitors.

Surprisingly, the present inventors have found a group of health supplements acting as protease inhibitor as tested in human cancer cell lines. These protease inhibitors of the present invention are believed to be strong nucleophilic activities and exhibit serine protease inhibiting properties either as single and/or in different combinations as confirmed in cell culture system. The present inventors have accurately analysis the inhibition activities and corelate with their chemical structures which definitely showed varying degrees of nucleophilic capabilities to compete with the serine amino acid at the site of scissile bond which is believed to be one the key players of enzymatic reactions. In the current invention, Inventors formulate the right combination of said health supplements in a tablet form which can be consumed orally. This inhibition is set forth more fully hereinafter.

Vitamins are an organic essential micronutrient that an organism needs in small quantities for the proper functioning of its metabolism (11-15). The term vitamin does not include the three other groups of essential nutrients: minerals, essential fatty acids and non-essential amino acids (16). Essential nutrients cannot be synthesized (17). in the organism, either at all or not in sufficient quantities, and therefore must be obtained from diet. Vitamin C can be synthesized by some species but not by human being. Vitamins have diverse biochemical functions such as a regulator of cell and tissue growth and differentiation (Vitamin A), hormone-like function, regulating mineral metabolism for bones and other organs (vitamin D), as enzyme cofactors and/or coenzymes or the precursors (Vit-B complex) and as antioxidants (Vit-C and E) (18,19). Both deficient and excess intake of a vitamin can potentially cause clinically significant illness, although excess intake of water-soluble vitamins is less likely to do so.

Almost all vitamins were discovered and/or identified between 1913-1948 and starting as commercially available products (tablets) from yeast-extract as vitamin B complex and semi-synthetic vitamin C in 1938 (20). From 1950s a mass production and marketing of vitamins supplements, including multivitamins, became available world-wide to prevent vitamin deficiencies in the general population (21).

Our invention is directly related with the several combinations of water-soluble vitamins and are available in market, showed wonderful inhibitory effects on serine proteases to a level of recommended doses vitamins used world-wide. Hence our invention showed an additional health benefits of these supplements on top of WHO and/or FDA recommended effects of vitamins in human health.

Any types of inflammatory reactions which imbalance the haemostatic status could be addressed using our invented inhibitors at the level of WHO recommended doses. The uniqueness of our invented inhibitors has less side effects due to their water-solubility and easy elimination process from body. So, during severe inflammatory process even excess doses could be recommended without major side-effects.

Inhibition of collagenase by our invented inhibitors could be another application for the treatment of arthritis as well as fibromyalgia, an inflammatory process in the junction of muscular tissue which is very common among elderly women.

A further proposed application of our invented inhibitors as detected in cell culture system against trypsin and elastase could be an additional benefit that can eliminate an excess damage of pancreatic tissue during inflammatory process and hence reduce the chance of development of pancreatic cancer and diabetics (22-23).

### BRIEF DESCRIPTION OF DRAWINGS

FIG. 1 Shows that in trypsin catalytic triad the His-57 is unprotonated and in the resting state. The amino acid Asp 102 helps His57 in correct conformation by accepting H+ so that it accepts H+ from Ser195 which then attacks the carbonyl carbon of the scissile peptide bond of peptide to initiate catalysis of the enzymatic reaction.

FIG. 2 Shows the representation of the transition state between the initial attack of Ser195 on the carbonyl carbon of the substrate and the tetrahedral intermediate. The catalytic triad functions as a charge relay system, as indicated by the distribution of partial charges.

FIG. 3 Chemical structure of Ascorbic acid (C)

FIG. 4 Chemical structure of Pyridoxine (B6)

FIG. 5 Chemical structure of Thiamine (B1)

FIG. 6 a) Shows the normal trypsin catalytic mechanism which is initiated by donating and accepting of hydrogen atoms by histidine amino acid to aspartate and serine amino acids respectively. This exchange of ions in catalytic triad follows further steps of catalysis process. b) explain the role of blocking effect of Ascorbic acid due to its Nucleophilicity which can easily donate hydrogen atom from position 4 and form a bond with amine group (NH2) of histidine and hence block the serine to attack on carbonyl carbon of the peptide bond and eventually stop further step of catalytic process.

FIG. 7 Showing the inhibitory effects of health supplement vitamin C, against serine proteases (SPs): Trypsin (T) and Elastase (E). Comparing with cell control (CC) [without serine protein protease and inhibitors] and wells with enzymes control trypsin (TC), Elastase (EC). Serine proteases (Trypsin and Elastase) treated with vitamin C showed inhibitions of enzymatic activities.

FIG. 8 This figure showed the microscopic morphological characteristic of A549 cells (4×10) following staining with crystal violet stain. Cells treated in serine protease inhibitory wells showed intact cell outlines and strongly attached in well surface (A, B) while the serine proteases (Trypsin and Elastase) showed the maximum removal of cells from well and those remained showed round shaped morphology compared (C, D) to cell control well (F) where all cells remained attached with plate surface with normal cell outlines.

FIG. 9 Showing the inhibitory effects of health supplements such as vitamin C and the combination of C (Ascorbic acid), B1 (Thiamine) and B6 Pyridoxine) labelled as tbc against serine protein proteases (Trypsin and Elastase). In comparison with controls (cell [CC] and enzyme (TC) controls) both preparations either ascorbic acid alone and its combination with Thiamine and Pyridoxine showed good level of inhibition against trypsin and elastase labelled as T+C, T+tbc and E+C, E+tbc respectively.

FIG. 10 This figure showed the morphological characters of A549 cells (4×10) following treatment of serine proteases (Trypsin and Elastase) with two formulations of inhibitors as mentioned in FIG. 9. Cells in serine protease inhibitory wells showed intact cell outlines and strongly attached with the well surface (A, B, C & D) while the serine proteases-trypsin (TC) control wells showed the maximum removal of cells from well surface and those remained showed round shaped morphology compared to cell control well (CC) where all cells remained attached with plate surface and having normal cell morphology.

FIG. 11 Different health supplements were used at five-fold dilutions against 0.125% trypsin as inhibitors and showed varying degrees of inhibitory effects as compared to cell and trypsin control wells (CC and TC respectively). Ascorbic acid at two formulations (AC-1 & AC-2) showed complete inhibition of trypsin catalytic activities up to 1:40 dilution (0.25%) while B1 and B6 inhibited trypsin up to 1:10 dilution (1.0% and 0.5%) respectively. However, the combination of these three components (tbc) showed 4 to 8× higher performances compared to single preparations.

FIG. 12 Shows the serine protease activity of Ascorbic acid (AC-1 & AC-2) against trypsin at 1.25% concentration. AC-1 efficiently neutralized the catalytic activities of trypsin at a concentration of 0.25% and started losing the capability of neutralization from 0.125% FIG. 13 Cell morphologies observed under microscope at different dilution of B1 as trypsin inhibitor compared to cell and trypsin control. The end point of inhibitory effects of B1 was found at dilution 1:10 (1.0%) and the inhibitory effects almost lost from dilution 1:20 to onward while B6 showed similar activities at 0.5% concentration.

FIG. 14 Cell morphologies observed under inverted microscope at different dilutions of combination of formulation (tbc-1 and tbc-2) using three separate serine protease inhibitors as shown in FIGS. 12 & 13 showed better performance which is almost 4× higher than AC and 8× higher than B1 and B6. This experiment showed the synergistic effects of the combination of three health inhibitors and was considered to be used as a tablet form for future application.

FIG. 15 Cell morphologies of cells under inverted microscope at 4×10 magnification shows that cells in Trypsin (TC) control well completed washed out during crystal violet treatment and PBS wash while cell control (CC) showed good morphological outline and remained nicely attached with surface.

FIG. 16 Showing the potential application of our invented serine protease inhibitor by inhibiting two potential serine protease enzymes: Furin and TMPRSS2 which are essential for the entry of SARSCOV2 into the host susceptible cells.

FIG. 17 Two serine protease enzymes (Furin and TMPRSS2) playing very crucial role in the entry process of SARSCOV2 into susceptible cells, however, Furin is also playing important role in the multiplication process of virus in the infected cell cytoplasm. Our invented serine protease inhibitor can block these enzymes at the entry process of virus as well as in the multiplication cycles of virus inside infected cells, hence reduces the pathogenesis of infection at the early stage of infection.

FIG. 18 Shows the simplified model of the serine protease system. Depiction of the complex interplay between the coagulation/fibrinolysis cascades and the complement system. The serine proteases of the complement, coagulation, and fibrinolysis systems are all highlighted in yellow. The black dotted arrow bars show previously known interactions of these systems. The red arrows identify the new paths of complement activation by the coagulation/fibrinolysis factors resulting in the generation of C3a and C5a. aPC, activated protein C; MAC, membrane attack complex; MBL, mannan-binding lectin; PK, prekallikrein

### DETAILED DESCRIPTION OF THE INVENTION

References made in detail to the presently preferred embodiments of the invention, which, together with the following examples, serve to explain the principles of the invention.

The serine protease inhibitors of the present invention are chemical structures with free electrons to donate during protein degrading enzymatic reactions.

The protease inhibitors of the present invention are health supplements easily available in market and have not to loss of activity when exposed to many proteolytic enzymes, including trypsin and pancreatic elastase. These inhibitors can also be used in combinations without losing their ability to form a stronger inhibitor effects on serine proteases such as trypsin and elastase as noticed in cell lines. The donation of necessary electrons from our invented inhibitors successfully blocks the serine amino acid in donating of electron in the catalytic pocket and hence prevent the digestion of intercellular bridges of cells.

The protease inhibitors of the present invention have been collected from pharmacy and/or in pure form from research laboratories. For the purposes of the present application, “pure form” or “purified form,” when used to refer to the protease inhibitors disclosed herein, shall mean substantially free of other chemicals which are not serine protease inhibitors. Preferably, the protease inhibitors of the present invention are at least 90% pure and preferably 95% pure.

The protease inhibitors of the present invention may be obtained in a pure form from laboratories and/or in combinations by the method comprising:
collecting either in tablet and/liquid forms from pharmacy
tablets are dissolved in distilled water into the desired concentration and filtered by 0.2 μm syringe
for the pure form, powders were collected from pharmaceutical research laboratories and then dissolve in distilled water and filtered by 0.2 μm syringe filter

In a preferred embodiment, supplements are diluted two to five-fold dilutions (where ever necessary) in PBS (pH 7.4) either as single component and/or various combinations and tested in cell lines grown in either 6, 12, 24 and/or 48 well plates. Cell (CC) and trypsin control (TC) were kept as protease negative and positive indicators in every experiment. Trypsin-EDTA (Gibco Lot no-2455123, Canada) was added into all wells (0.125%) expect cell control wells. Incubate the plate at 37° C. until 95%-98% cells were detached and/or separated from each other as observed under microscope in trypsin treated well.

Fluids from the plate was discarded and washed twice with PBS. Following wash the plate was stained with 1% crystal violet in ethylene at room temperature for 5 minutes. Following incubation, crystal violet from each well was discarded and washed two to three times with PBS and removed any excess dye from each well. Following drying at room temperature plates were observed for cell layer and its morphological outlines under inverted microscope for the comparative analysis of the inhibitory effects of proposed inhibitors compared to control wells [trypsin control (TC) and cell control (CC)].

Protease inhibitors identified by the above method generally exhibit competitive activity in relation to the serine protease, such as trypsin and elastase by competitive activity, it is meant that each molecule of protease inhibitor will react with and thereby significantly decrease the activity of one molecule of trypsin and/or elastase (SIGMA, cat. No-E017-5 mg) in terms of its nucleophilic activities of serine amino acids which is the key player of donating electron of initiation of protease activity. In a solution of the preferred inhibitor of the present invention in which the inhibitor and trypsin are present in a concentration greater 0.355 mM verses 0.524 mM such inhibitor would react >95% of an equivalent molecular amount of trypsin.

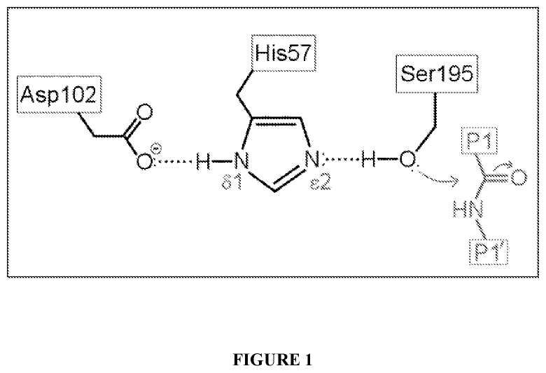

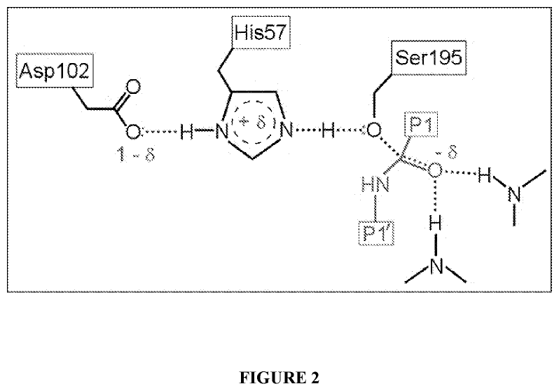

As noted above, the present inventors have succeeded in detecting of serine protease inhibitor from commercially available health supplements and/or in purified form collected from pharmaceutical laboratories. Selection of such supplements in a purified form was a prerequisite step to the correct combination of the inhibitors which either acts as competitor and/or blocking agent of serine amino acid as potential electron donor (nucleophile) in the catalytic triad (serine-histidine-aspartate) as shown in FIG. 1.

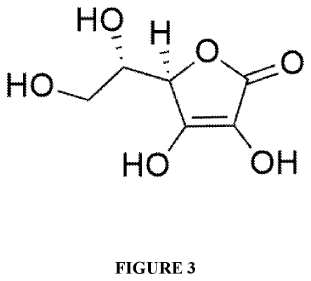

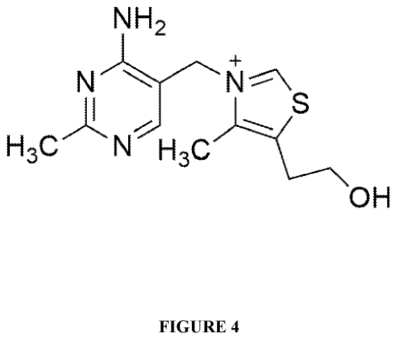

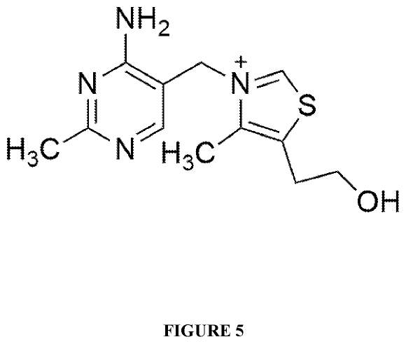

Preferred protease inhibitors of the present invention are Ascorbic acid, Thiamine and Pyridoxine as shown in FIGS. 3-5.

It has been found that the protease inhibitors according to our invention have more than one nucleophilic side depending of their chemical structure. By more than one distinct potential nucleophilic side it is meant that inhibitors have multiple active sites which could be functional against different enzymes. The presence and location of these active sites have been determined by the active nucleophilic sites in chemical structures of the protease inhibitor. It is believed that the presence of distinct nucleophilic sites confers on the instant protease inhibitors ability to inhibit a variety of serine proteases such as trypsin, elastase, collagenases and even peroxidases.

It has been further noted that, due to the plurality of distinct nucleophilic sites of these protease inhibitors, the protease inhibitors may serve as frameworks on which various other active sites may be constructed to create protease inhibitors having additional properties. The preferred embodiment of the present invention is protease inhibitors those either as alone and/or in various combinations inhibit protease such as trypsin and elastase. Trypsin is a member of a class of proteases known as serine proteases that share a common mechanism of action and many structural features.

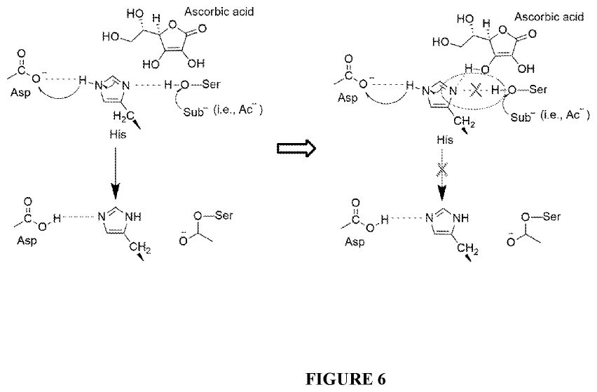

Our invented protease inhibitors are non-proteins and are totally chemical based FDA approved health supplements and believed to work at the subatomic levels where ionic exchanges take place between nucleophilic and electrophilic sites in serine and histidine amino acids in the catalytic triad of trypsin binding pocket once substrate activates the triad (FIG. 6). Ascorbic acid has nucleophilic activity and can donate two H+ from positions 3 and 4 (23) and could be the strong competitor of serine amino acid in donating H+ to histidine.

Using of such supplement alone and/or various combinations will be well tolerated by human physiology and have little or no chance of immunological disturbance which could be possible for the protein and/or peptide-based serine protease inhibitors.

Furthermore, it is contemplated that various combination of such health supplements, may enhance either the trypsin inhibitory properties or other serine proteases inhibitory properties or of the present protease inhibitors while meet up other essential requirements of body as micro-nutrient supplements. The protease inhibitory may also be separated into separate supplement, each of which retains a desired inhibitory function as well normal body requirement. The present claims extend to other inhibitors produced by these means.

While the above-stated inhibitors yield a trypsin inhibitor which is preferred and has the above enumerated characteristics, the present invention also provides synergistic effects of such combinations which exhibit stronger inhibiting activity of trypsin in cell culture system which are also desirable from a pharmaceutical standpoint of view. Accordingly, such analogs are also preferred compositions of the present invention. In particular, if ascorbic acid mixed trypsin and elastase the resultant inhibitor would exhibit improved resistance to oxidative inactivation and, therefore, exhibit improved trypsin inhibitor and other enzymatic properties.

The protease inhibitor of the present inventions is contemplated for human and veterinary uses in the form of pharmaceutical supplements possessing serine protease inhibitor activity, particularly trypsin inhibiting activity. It is expected that combination formulation containing, as at least one of the active ingredients, one of the present serine protease inhibitors would also contain appropriate, pharmaceutically acceptable carriers, diluents, fillers, binders and other excipients depending on the dosage form contemplated. It is also contemplated that pharmaceutical preparations containing the serine protease inhibitors, particularly trypsin and/or other serine protease inhibitor proteins, of the present invention can be administered locally, as by injection or oral application for treatment of localized and/or systematic enzyme imbalances. The present inhibitors, particularly leukocyte elastase inhibitor, may also be administered as an aerosol, particularly when applied to the lungs as in the treatment of emphysema. In these situations, suitable carriers, diluents and propellants would be used.

Our invented protease inhibitors are currently in use as health supplements, however, during the amount of the protease inhibitor to be administered would be dependent in each dosage situation on the amount of excess proteolytic enzyme present produced during pathological conditions. To determine the appropriate dosage, the excessive amount of proteolytic enzyme present and would determine, based on the activity of the particular species of protease inhibitor or mixtures thereof to be administered, the total amount of protease inhibitor necessary to neutralize the excess proteolytic enzyme. However, it should be noted being water soluble nature that large excesses of the protease inhibitors of the present invention would not be toxic or cause an adverse reaction when administered to an organism with an excess of proteolytic enzymes and will be eliminated with body fluid preferably by urine. As such, it is preferred that an amount in excess of the optimal amount of protease inhibitor be administered, rather than a lesser amount than the optimum. However, we will follow the WHO recommended doses for its future application preferably in oral as tablet.

Examples of the products of the present invention and representative processes for their isolation and manufacture appear in the following examples.

EXAMPLE-01: Screening of protease inhibitors in cell lines derived from human carcinoma cell lines (A549)

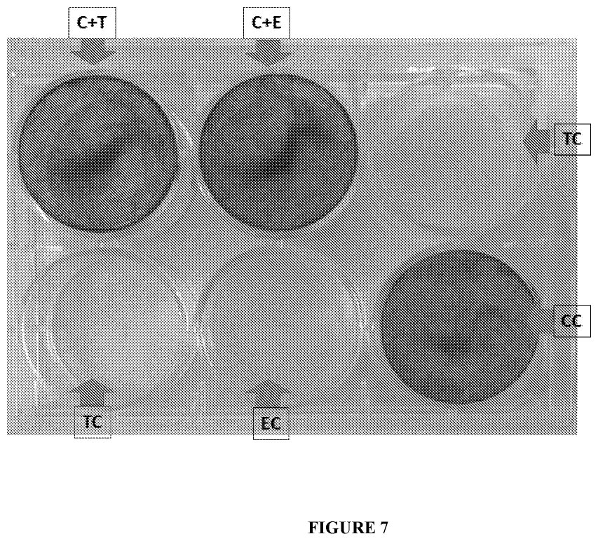

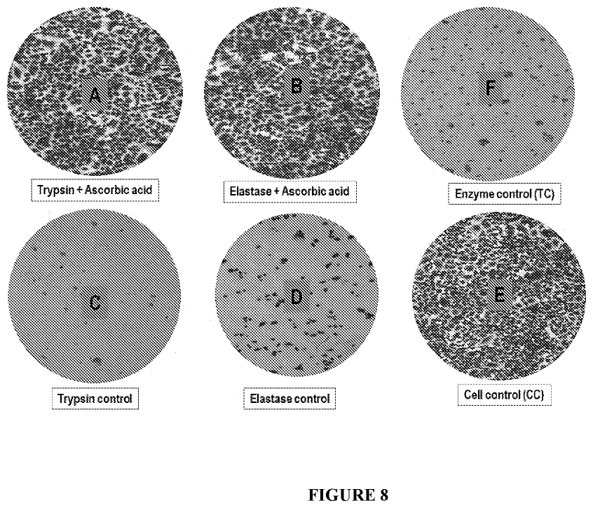

Health supplements were collected from pharmaceutical laboratory and were randomly screened in one of the lungs cancer cell lines called A549 for their protease inhibitory effects. Cells were cultivated in 6 well plate using standard technique until confluent. Once the plate is ready, culture media from each well were removed and washed two times with PBS. Following wash, 500 μl of supplements at appropriate dilutions were added into well followed by 500 μl of trypsin (0.25%) to each well except the well having cell control (CC). Plate was incubated at 37° C. and checked at every 5 minutes to see the effect of trypsin on cell morphology. Once cells in trypsin control well lift off from the surface, discard fluid from all wells, wash two times with PBS and stained cells by 1% crystal violet (in alcohol) for two-three minutes. Following staining wells were washed 2-3 times with PBS in order to remove excess dye and finally checked the cell morphology using inverted microscope as well as by naked eye (FIG. 7, 8).

Example-02: Screening of using different formulation of inhibitors against serine protease (Trypsin and Elastase) in A549 cell lines.

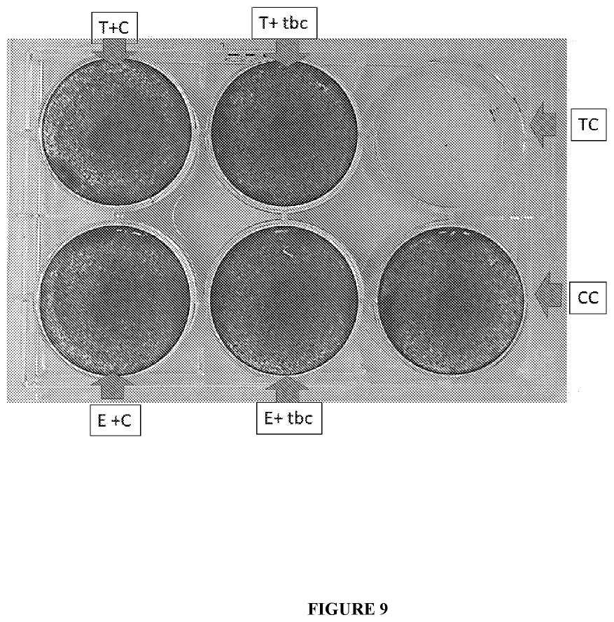

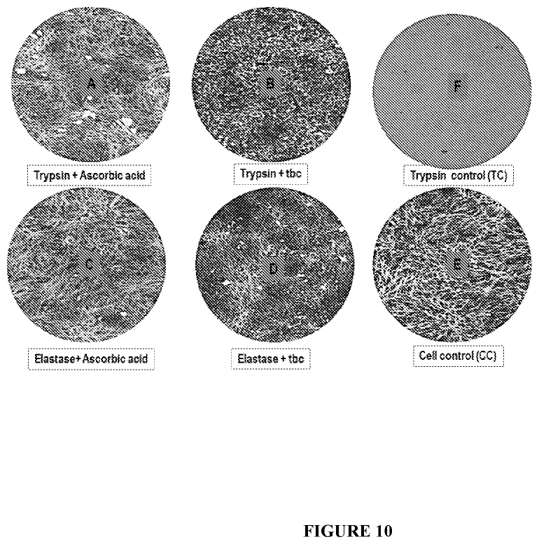

A further experiment was conducted using serine proteases (Trypsin and Elastase) against two formulations of health supplements such as Vitamin C and combination Vitamins C (PC Drug Center Ltd, Bangkok, Thailand) with Thiamine (B1-Roche, lot no: 8077395)) and Pyridoxine (B6, Roche, lot no: 8079496). This formulation of three vitamins is designed as tbc and experiment was conducted in six well plate with confluent A549 cells using technique described in [0044]. Results of the experiment are showed in FIGS. 9 & 10. For the inhibitory treatment 2.5% of Ascorbic acid with 1.25% of trypsin and 0.5% of elastase was used while tbc formulation (ascorbic acid-1000 mg, pyridoxine-100 mg and thiamine-50 mg per tablet) used against trypsin and elastase were Ascorbic acid (2.5%), Thiamine (0.1% and Pyridoxine (0.15%) respectively.

EXAMPLE-03: Titration of serine protease inhibitors against serine protease (Trypsin) in cell culture system.

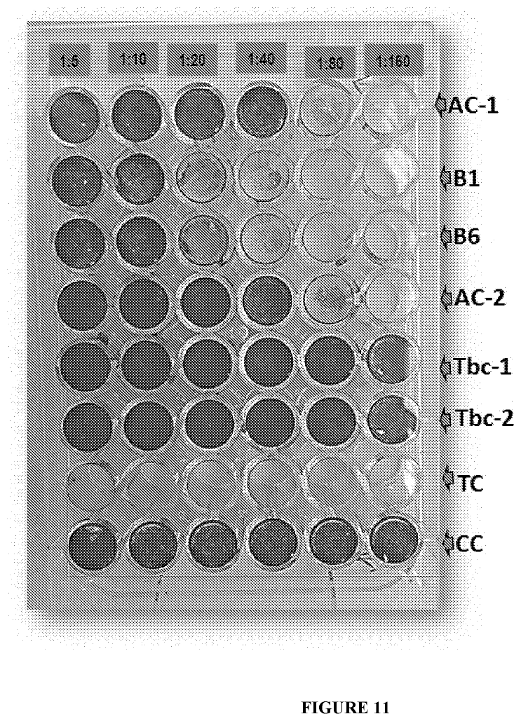

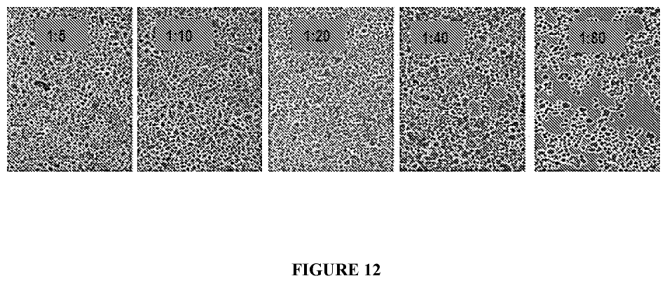

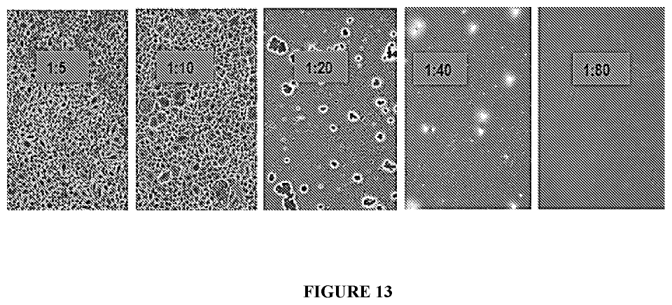

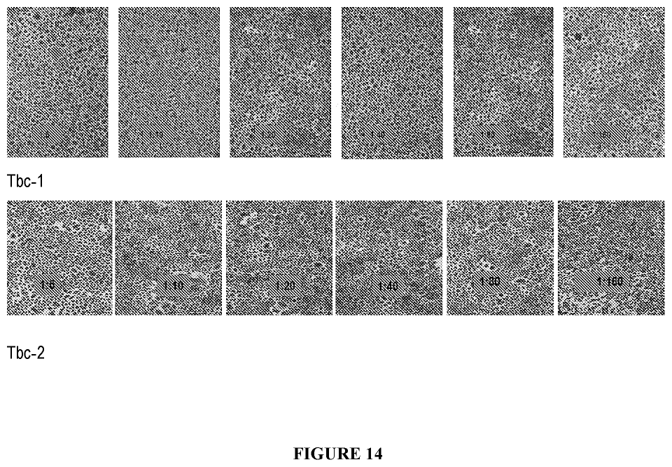

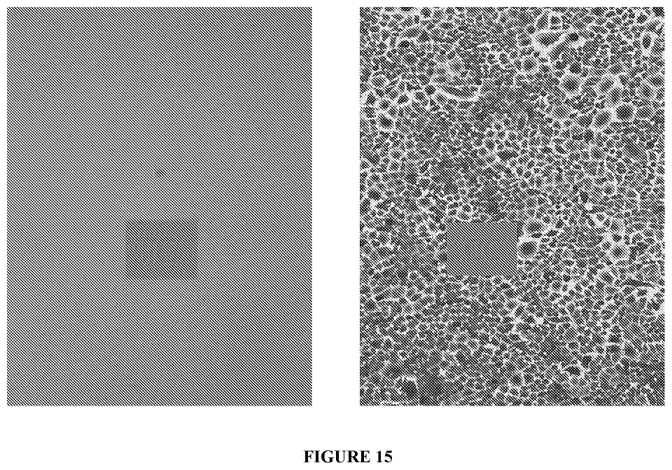

Three formulations of proposed serine protease inhibitors were used All supplements were collected in a chemically purified powder forms from the pharmaceutical laboratory of Faculty of Pharmaceutical Sciences, PSU and conducted similar experiment at five-fold (5) serial dilutions in 24 well plate to determine the end point of each supplement as trypsin inhibitors (FIG. 11). The end point of trypsin inhibition of each supplement was calculated in terms of μg/ml. The cell morphologies were also observed by under inverted microscope (4×10) and are shown FIG. 12-15.

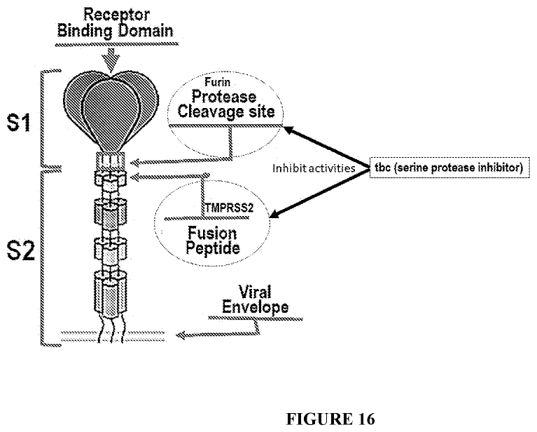

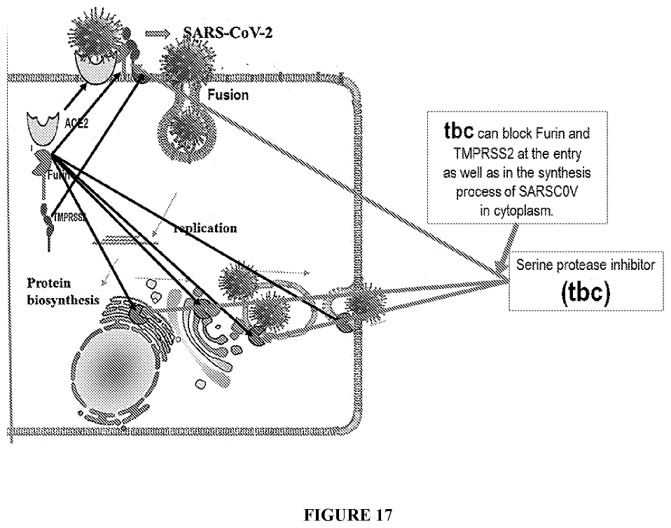

EXAMPLE-04: Potential application of the invented serine protease in a tablet form through oral route could be applied in any form of intense inflammatory diseases and/or pathological conditions. Our invented serine protease inhibitor has a great chance in the management of COVID-19 infection in both ways: viral and host. Our formulated and invented Inhibitor will be able to prevent the viral entry into susceptible cells by inhibiting two serine protease enzymes known as Furin and TMPRSS2 which are the key enzymes to cut the spike protein into S1 and S2 and fusion of S2 with cell membrane that facilitate the entry of virus into cells (FIG. 16). Our invented inhibitor can also prevent the multiplication process of virus inside the cell cytoplasm by inhibiting Furin, a serine protease group of enzymes. In terms of host factor the severity of COVID-19 is found to be associated with intense inflammatory process which could be due to massive complement activation due to antigen-antibody complexes at the later stages of infection. There are at least three (3) serine protease enzymes actively involved in complement cascade reaction such as C1qr, C3 convertase and C5 convertase and our invented inhibitors are uniquely active to inhibit such enzymes and hence keep the inflammatory process under control (FIG. 17).

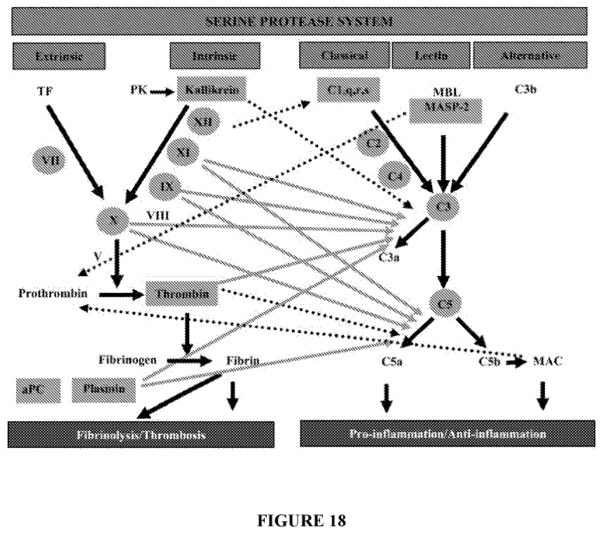

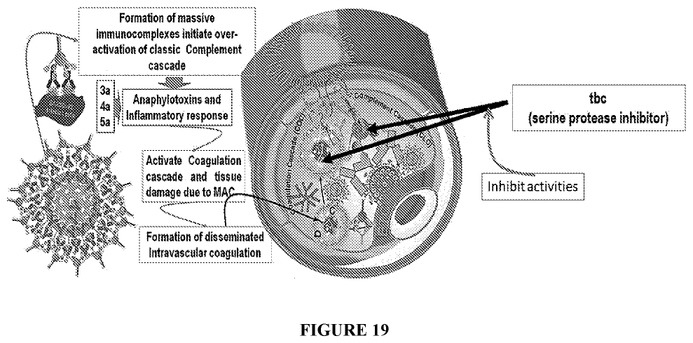

Another devastating pathological condition developed among severe COVID-19 cases is the formation of disseminated intravascular coagulation known as DIC. This process is also orchestrated by a number of serine proteases as well as complements (24, 25). So, our invented inhibitor could be the powerful tool to bring the pathological condition under control (FIG. 18, 19).

### SUMMARY OF THE INVENTION

An object of the present invention is to provide the detection of inhibitory activities of health supplements which are active against one or a combination of a varieties of inflammatory enzymes including serine proteases

Further objective of the present invention includes providing purified forms of protease inhibitors which are available as health supplement preparation and exhibiting activity against inflammatory enzymes such as serine proteases in cell culture system. Furthermore, the identification of active components of such a protease inhibitor as claimed herein (Protease inhibitors according to the invention) with enhanced or equivalent properties in several combinations is also one of the objects of the invention.

Additional objects and advantages of the invention will be set forth in part in the description which follows, and in part will be obvious from the description or may be learned from practice of the invention. The objects and advantages may be realized and attained by means of the biological/chemical activities and/or instrumentalities combinations particularly pointed out in the appended statements.

To achieve the objects and in accordance with the purposes of the present invention, a protease inhibitor is disclosed which exhibits inhibitory activity against serine proteases, particularly trypsin and elastase. The preferred inhibitors have been collected from pharmacy as well as pharmaceutical laboratories in a purified form. The protease inhibitors according to the invention have the ability as individual and/or in combinations of capable of expression of the desired serine protease inhibiting activity in the absence of any biochemical stimulus in cell culture system.

The inhibitor of the present invention is a chemically purified health supplements of serine protease inhibitor consisting of a one or more nucleophilic activities which is claimed as inhibitor and capable of inhibiting the protease activity against at least one serine protease. Thus, particularly preferred inhibitors according to the present invention have the nucleophilic activity.

Preferably, the serine protease inhibitor activity exhibited equivalent inhibition to that of the commercially available inhibitor and being used as health supplements at micro-nutrient level and being tested and approved by health regulatory bodies such FDA, WHO, EEC etc. Thus, the present invented serine inhibitor activity of health supplements has a little and/or no health hazardous effect at recommend dose and will be well accepted by people and regulatory bodies worldwide.

Our invented health supplements will provide dual activities such as serine protease inhibitors on top of other health benefits which already recommended by health authorities and being established by medical sciences.

Current inhibitors have potential applications to minimize the over activities of serine proteases during inflammatory process including the current pandemic of COVID-19.

Uncontrolled Serine proteases activities lead to excessive complement and thrombosis activities followed by severe inflammation and the formation of disseminated intravascular coagulation (DIC) could be controlled by our current invented inhibitors, as these pathological conditions utilize serine proteases at different level conversion.

Our proposed formulation and combination of three inhibitors (Vitamin C, B1 and B6) in a tablet form could be the right choice of delivery as serine protease inhibitors through oral route.

### BEST MODE FOR CARRYING OUT THE INVENTION

As already mentioned in the full disclosure of invention.

1. A serine protease inhibitor consisting of a combined formulation of health supplements selected from the group consisting of chemically purified ascorbic acid, thiamine and pyridoxine.

2. A serine protease inhibitor according to claim 1, wherein the serine protease inhibitor inhibits the protease activity of more than one serine protease.

3. A serine protease inhibitor according to claim 1, wherein one or more combination of said supplements is more powerful in inhibiting specific serine proteases such as trypsin and elastase.

4. A serine protease inhibitor according to claim 1, wherein said inhibitor is more active in inhibiting trypsin than any single component of the formulation.

5. A serine protease inhibitor according to claim 1, wherein one or more of the selected from the group of health supplements and said inhibitor is more active in inhibiting the native serine protease inhibitor in powder or liquid and/or tablet form.

6. A pharmaceutical composition comprising the serine protease inhibitor according to claim 5 which is in tablet form, comprising: ascorbic acid (500 mg-1000 mg), pyridoxine (100 mg-150 mg) and thiamine (50 mg-100 mg) per tablet and/or any combination suitable for medical application.

7. The composition as claimed in claim 4 which is in a form suitable for administration by a route selected from the group consisting of oral administration, parenteral administration, local administration and aerosol administration.

8. Use of the inhibitors as claimed in claim 1 for preparing a medicament for inhibiting proteolytic enzymes in an organism.

9. A process for preparing a serine protease inhibitor, consisting of a multiple chemically pure FDA approved health supplements commonly available in market, said serine proteases inhibitor inhibiting the protease activity of at least one serine protease in cell culture system is comprising
(a) collecting chemically health supplements;
(b) formulation of different form of combinations
(c) identifying the inhibitory activities against serine protease such as trypsin and other serine proteases (such as elastase) in cell culture system, and
(d) formulation the best combination of inhibiting the serine protease inhibiting activities in cell culture system referred.

10. A process for preparing a pharmaceutical composition comprising the serine protease inhibitor as claimed in claim 1, wherein a pharmaceutical acceptable carrier by combining the components in a manner known per se and can be used for the management and/or treatment of patients with intense inflammatory conditions either due to infection and/or other pathological conditions like cancer.

11. The serine protease inhibitor according to claim 10, wherein the inhibitor can either be used along and/or in combination with other drugs.

12. The serine protease inhibitor according to claim 11, wherein our formulated combined inhibitors can also be used as health supplements to lead a healthy life without infection and/or pathological conditions.
